(19) United States
(12) Patent Application Publication
(10) Pub. No.: US 2024/0122711 A1
(43) Pub. Date: Apr. 18, 2024

(54) SYSTEM AND METHOD FOR REDUCING TRICUSPID REGURGITATION

(71) Applicant: EDWARDS LIFESCIENCES CORPORATION, Irvine, CA (US)

(72) Inventors: Michael W. Sutherland, Pelham, NH (US); Steven D. Cahalane, Pelham, NH (US)

(21) Appl. No.: 18/392,853

(22) Filed: Dec. 21, 2023

Related U.S. Application Data
(63) Continuation of application No. 17/083,127, filed on Oct. 28, 2020, now Pat. No. 11,890,193.
Application No. 17/083,127 is a division of application No. 15/393,809, filed on Dec. 29, 2016, now Pat. No. 10,828,160.
(60) Provisional application No. 62/272,871, filed on Dec. 30, 2015.

(51) Int. Cl. A61F 2/24; A61B 17/04

(52) U.S. Cl. CPC A61F 2/246; A61B 17/0401; A61F 2/2451; A61B 2017/00867

(57) ABSTRACT

A method of treating a tricuspid valve that defines an annulus between a right atrium and a right ventricle of the heart is described. A first anchor and a second anchor are transluminally advanced to the heart. The first anchor is anchored inside a coronary sinus of the heart. The second anchor is anchored, from the right atrium, at a site that is across the annulus from the coronary sinus by driving a distal portion of the second anchor into the annulus at the site. Subsequently, a distance between the first and second anchors is reduced by applying tension to a tensioning member coupled to the first and second anchors. The tension is maintained by locking a lock member to the tensioning member. Other embodiments are also described.

### CROSS REFERENCE TO RELATED APPLICATION

The present application is a continuation of U.S. Ser. No. 17/083,127 to Sutherland et al., filed Oct. 28, 2020, which published as US 2021/0038378, and which is a divisional of U.S. Ser. No. 15/393,809 to Sutherland et al., filed Dec. 29, 2016 (now U.S. Pat. No. 10,828,160), which claims priority to U.S. patent application Ser. No. 62/272,871, filed Dec. 30, 2015. The foregoing applications are hereby incorporated by reference herein in their entirety.

### TECHNICAL FIELD

The present teachings generally relate to tissue anchor systems and uses thereof in treating tricuspid regurgitation.

### BACKGROUND

Tricuspid valve diseases relate to conditions in which the valve between the two right heart chambers (i.e., the right ventricle and the right atrium) doesn't function properly and these diseases often occur with other heart valve problems. Examples of tricuspid valve diseases include tricuspid valve regurgitation, tricuspid valve stenosis, tricuspid valve atresia, and the Ebstein's anomaly. In the tricuspid valve regurgitation, the tricuspid valve doesn't close properly and blood flows back into the right atrium; in the tricuspid valve stenosis, the tricuspid valve is narrowed and reduces the amount of blood flowing into the right ventricle; in the tricuspid atresia, a congenital heart disease, a solid wall of tissues blocks the blood from flowing between the two right heart chambers; and in the Ebstein's anomaly, a malformed tricuspid valve situates at a position lower than the normal position in the right ventricle and causes blood to flow back into the right atrium. There are other tricuspid valve diseases generally known to a person with ordinary skill in the art and these tricuspid valve diseases are also included in the present teachings.

A tricuspid valve disease can be corrected by an annuloplasty ring. In some instances, this device is preferred for surgically repairing a defective tricuspid valve. An annuloplasty ring is an anatomically-correct three-dimensional (3D) ring and can flexibly conform to the heart valve opening. This ring is implanted into a defective tricuspid valve and reduces the valve opening. Properly implanted, an annuloplasty ring allows the valve to open and close properly.

Tricuspid valve repair surgeries can be done in one of the following two ways: a minimally invasive surgery or an open-heart surgery. A minimally invasive method involves making a small incision in the upper or lower chest and inserting a valve repairing system/device percutaneously. After the valve is repaired, the incision may be closed with dissolving sutures. In some minimally invasive techniques the initial incision or puncture can be allowed to heal without sutures. In this event any bleeding is stopped with manual compression following the procedure. Compared to an open-heart surgery, advantages of a minimally invasive approach include a shorter recovery time, less post-operation pain, and earlier return to work and normal daily activities.

However, there are drawbacks in either procedure and therefore needs still exist for repairing a diseased tricuspid valve with alternative approaches.

### SUMMARY

One aspect of the present teachings provides a percutaneous repair system for reducing a tricuspid annulus circumference. The system comprises a first anchor configured to be deployed inside the coronary sinus, and a second anchor configured to be deployed across the tricuspid annulus. The system further comprises a first tensioning member configured to apply tension to the first anchor with a fixed end connecting to the first anchor and a free end; and a second tensioning member configured to apply tension to the second anchor with a fixed end connecting to the second anchor, and a free end. The system further comprises a lock member joining the free ends of both first and second tensioning members. The lock member is configured to maintain the tension on both the anchors.

Another aspect of the present teachings provides a percutaneous repair system for reducing a tricuspid annulus circumference. The system comprises a first anchor configured to be deployed inside the coronary sinus, and a second anchor configured to be deployed across the tricuspid annulus. The system further comprises a first tensioning member configured to apply tension to the first anchor with a first end connecting to the first anchor and a second end joining a lock member. The second anchor is configured to slide over the first tensioning member. The first tensioning member is configured to apply tension to the first anchor, and the lock member is configured to maintain the tension.

Another aspect of the present teachings provides a percutaneous repair system for reducing a tricuspid annulus circumference. The system comprises a first anchor configured to be deployed inside the coronary sinus and a second anchor configured to be deployed across the tricuspid annulus. The system further comprises a first distance between the first and second anchors and a second reduced distance between the first and second anchors.

Another aspect of the present teachings provides a method for percutaneously reducing the circumference of a tricuspid annulus. This method comprises implanting a first anchor into the coronary sinus and implanting a second anchor across the tricuspid annulus. In various embodiments, each of the first and second anchors connects to a tensioning member. The method further comprises applying tension to both the tensioning members so as to reduce the distance between the first and second anchors from a first distance to a second distance. The method further comprises implanting a locking member to secure the second distance between the first and second anchors. The method further comprises reducing the circumference of the tricuspid valve by tensioning both the tensioning members.

### BRIEF DESCRIPTION OF THE DRAWINGS

FIG. 1 is a perspective view of an embodiment of the present teachings where a vascular anchor and an annulus anchor deployed at a treatment location, and tension is applied to both the anchors deployed across the tricuspid annulus in accordance with the present teachings.

FIG. 2 is a perspective view of an exemplary vascular anchor delivery system percutaneously inserted into the coronary sinus in accordance with the present teachings.

FIGS. 3A-3B are embodiments of an exemplary vascular anchor (12) in its delivery profile and attached to a delivery system in accordance with the present teachings.

FIG. 4 is an embodiment of an exemplary vascular anchor in its deployed profile in accordance with the present teachings.

FIG. 5 is a perspective view of an exemplary vascular anchor deployed inside the coronary sinus in accordance with the present teachings.

FIG. 6 is a perspective view of an exemplary wire positioned across the annulus in accordance with the present teachings.

FIG. 7 is a perspective view of an exemplary annulus anchor deployed inside the right ventricle in accordance with the present teachings.

FIG. 8 is an embodiment of an exemplary annulus anchor in its deployed profile in accordance with the present teachings.

FIG. 9A is an embodiment of an exemplary annulus anchor in its delivery profile and attached to a delivery system in accordance with the present teachings.

FIG. 9B is an embodiment of an exemplary annulus anchor in its deployed profile in accordance with the present teachings.

FIG. 10 is a perspective view of an embodiment of the present teachings where tension is applied to both a deployed vascular anchor and a deployed annulus anchor in accordance with the present teachings.

FIG. 11 is a perspective view of an embodiment of the present teachings where a deployed locking member joins both the tensioning members of a deployed vascular anchor and a deployed annulus anchor in accordance with the present teachings.

FIG. 12 is another perspective view of an exemplary positioning wire being placed across the annulus inside the right ventricle in accordance with the present teachings.

FIG. 13 is another perspective view of an exemplary annulus anchor deployed inside the right ventricle in accordance with the present teachings.

FIG. 14 is another perspective view of an embodiment of the present teachings where a locking member is deployed in accordance with the present teachings.

FIG. 15 is another perspective view of an exemplary vascular anchor deployed inside the right ventricle in accordance with the present teachings.

FIG. 16 is another perspective view of an exemplary vascular anchor deployed inside the right ventricle in accordance with the present teachings.

FIG. 17 is another perspective view of an embodiment of the present teachings where a locking member is deployed in accordance with the present teachings.

FIG. 18 is an embodiment of an exemplary vascular anchor in its deployed profile in accordance with the present teachings.

FIG. 19 is an embodiment of an exemplary vascular anchor in its deployed profile in accordance with the present teachings.

### DETAILED DESCRIPTION

Certain specific details are set forth in the following description and figures to provide an understanding of various embodiments of the present teachings. Those of ordinary skill in the relevant art would understand that they can practice other embodiments of the present teachings without one or more of the details described herein. Thus, it is not the intention of the Applicant(s) to restrict or in any way limit the scope of the appended claims to such details. While various processes are described with reference to steps and sequences in the following disclosure, the steps and sequences of steps should not be taken as required to practice all embodiments of the present teachings.

As used herein, the term “lumen” means a canal, a duct, or a generally tubular space or cavity in the body of a subject, including a vein, an artery, a blood vessel, a capillary, an intestine, and the like. The term “lumen” can also refer to a tubular space in a catheter, a sheath, a hollow needle, a tube, or the like.

As used herein, the term “proximal” shall mean close to the operator (less into the body) and “distal” shall mean away from the operator (further into the body). In positioning a medical device inside a patient, “distal” refers to the direction away from a catheter insertion location and “proximal” refers to the direction close to the insertion location.

As used herein, the term “wire” can be a strand, a cord, a fiber, a yarn, a filament, a cable, a thread, or the like, and these terms may be used interchangeably.

As used herein, the term “sheath” may also be described as a “catheter” and, thus, these terms can be used interchangeably.

Unless otherwise specified, all numbers expressing quantities, measurements, and other properties or parameters used in the specification and claims are to be understood as being modified in all instances by the term “about.” Accordingly, unless otherwise indicated, it should be understood that the numerical parameters set forth in the following specification and attached claims are approximations. At the very least and not as an attempt to limit the application of the doctrine of equivalents to the scope of the attached claims, numerical parameters should be read in light of the number of reported significant digits and the application of ordinary rounding techniques.

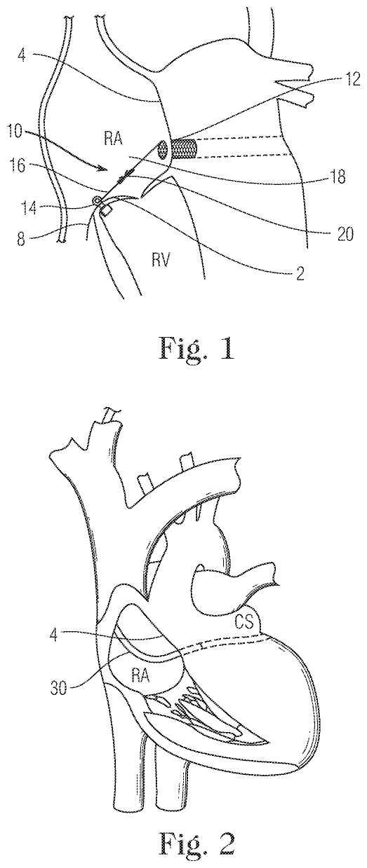

The present teachings relate to devices and methods for treating a tricuspid valve regurgitation percutaneously. Although referring to FIGS. 1 to 19, a person with ordinary skill in the art would recognize that the figures and description thereto refer to various embodiments of the present teachings and, unless indicated otherwise by their contexts, do not limit the scope of the attached claims.

An aspect of the present teachings relates to methods of reducing the circumference of a tricuspid valve (2). For example, now referring to FIG. 1, the circumference of a tricuspid valve (2) can be reduced by deploying an anchoring system (10) with a vascular anchor (12) deployed inside the coronary sinus and an annulus anchor (14) engaging at a location along the anterior or posterior annulus. Each anchor is attached with a flexible tensioning member (16, 18). Tension is then applied to both the anchors, and a lock member (20) secures free ends of both the flexible tensioning members (16, 18) while maintaining the tension at a pre-defined level. Upon deployment of this anchoring system (10), the tricuspid annulus (8) is pulled toward the inferior portion of the atrial septum (4). Depending on the location of the annulus anchor (14), a portion of the annulus could fold. Alternatively, the overall shape of the annulus could change. Both the results would lead to more coaptation of the tricuspid leaflets and, therefore, reduce or eliminate the tricuspid valve regurgitation jet. As a result, the tricuspid valve (2) could be fully closed during a right ventricular systole.

FIGS. 2-4 illustrate the deployment of a vascular anchor (12) inside the coronary sinus (CS) according to the present teachings. According to some embodiments, a vascular anchor delivery system (30) gains access through a standard right heart catheterization procedure, for example, through the femoral vein, the inferior vena cava, and the right atrium. Once inside the right atrium, the vascular anchor delivery system (30) extends further through the opening of the coronary sinus at the inferior portion of the atrial septum (4). In one embodiment, the vascular anchor (12) is then deployed inside the coronary sinus, 15-25 mm from its right atrium opening.

FIG. 2 illustrates an alternative embodiment of delivering a vascular anchor (12) inside the coronary sinus. In this embodiment, the access to the right atrium is gained by entering the jugular vein; extending through the right brachiocephalic vein and the superior vena cava; and reaching the right atrium. Once inside the right atrium, the delivery system further extends downwardly towards the inferior region of the atrial septum (4), and enters the opening of the coronary sinus. Similarly, once inside, a vascular anchor (12) is then deployed inside the coronary sinus.

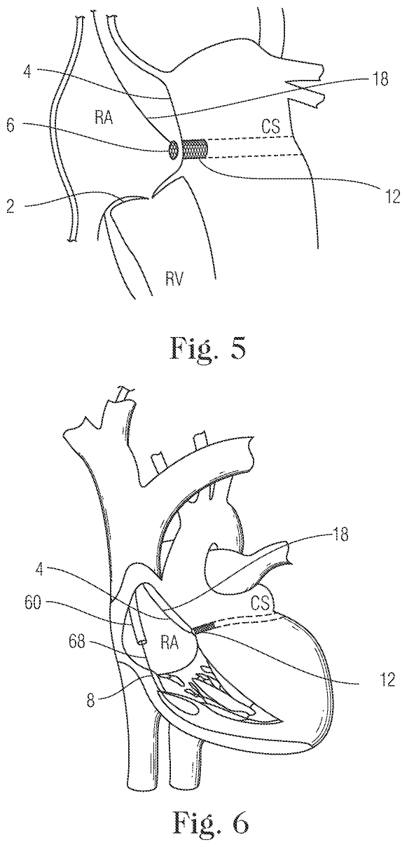

FIG. 5 illustrates a vascular anchor (12) deployed inside the coronary sinus, where the vascular anchor (12) secures itself to its surrounding tissue, and the vascular anchor (12) connects to a flexible tensioning member (18) which extends proximally outside of the coronary sinus. As shown in FIG. 5, a portion of the flexible tensioning member (18) is inside the right atrium, and the flexible tensioning member (18) further extends proximally with its proximal end extending outside of the body.

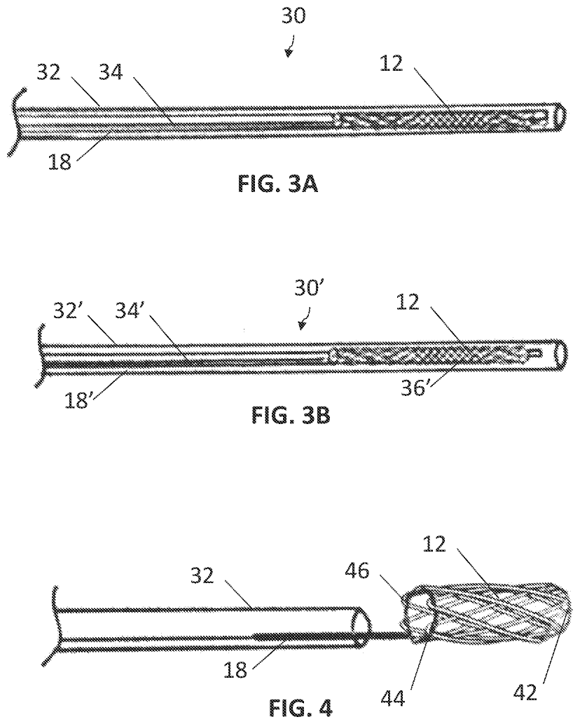

FIGS. 3-4 further illustrate an embodiment of the delivery system (30, 30′). The delivery system (30, 30′) includes a delivery sheath (32, 32′) that holds a vascular anchor (12) in its delivery profile. The delivery system (30, 30′) could further includes a mechanism (34, 34′) that can be used to transport a vascular anchor (12), for example from the proximal end of the delivery sheath (32, 32′) to the distal end of the delivery sheath (32, 32′), or push the vascular anchor (12) distally out of the distal end of the delivery sheath (32, 32′).

According to some embodiments of the present teachings, the vascular anchor (12) deployed inside the coronary sinus has an elongated body which is suitable for being positioned inside the vasculature. In some embodiments, the vascular anchor (12) is configured to collapse into a radially profile which is suitable to be delivered percutaneously by a delivery system (30, 30′) as described above, for example, as shown in FIGS. 3A-3B. In some embodiments, the vascular anchor (12) expands radially during the deployment. In some embodiments, as the vascular anchor (12) expands radially, the overall longitudinal length of the vascular anchor (12) shortens, for example, as shown in FIG. 4.

In some embodiments, the vascular anchor (12) is to be delivered through a vascular anchor delivery mechanism (34, 34′). The delivery mechanism (34, 34′) is designed to engage the vascular anchor (12) during its delivery, which allows the vascular anchor (12) to be pushed distally, pulled proximally, or held steady while the vascular anchor (12) transitions from its collapsed delivery profile into its radially expanded deployed profile. The engagement between the delivery mechanism (34, 34′) and the vascular anchor (12) is configured to be detachable, for example, at the distal end (42) of the vascular anchor (12), at the proximal end (44) of the vascular anchor (12), or through a longitudinal lumen (46) of the vascular anchor (12). The connecting flexible tensioning member (18) is configured to be disposed inside the delivery mechanism (34, 34′), or alternatively, along the delivery mechanism (34, 34′) while locating inside the delivery sheath (32, 32′).

In some embodiments, the vascular anchor (12) is made from a super-elastic or shape-memory material such as Nitinol. The super-elastic material would allow the vascular anchor (12) to be advanced to the implantation site in a collapsed configuration. Simply unsheathing the vascular anchor (12) could then allow the vascular anchor (12) to expose, expand, and contact the walls of the vasculature, for example, the coronary sinus. According to one embodiment of the present teachings, as shown in FIG. 3A, the delivery system (30) includes an outer sheath (32) and an inner catheter (34) which resides inside the lumen of the outer sheath (32). The inner catheter (34) resides inside the collapsed lumen of the vascular anchor (12) and is configured to engage the vascular anchor (12) when the vascular anchor (12) is in its collapsed state by a known mechanism to those skilled in the field, such as a simple hook or groove mechanism. In some embodiments (not shown), the inner catheter (34) features an inner lumen which is configured to allow a positioning wire to pass through the inner lumen. In some embodiments, the inner lumen of the inner catheter (34) is configured to allow a 0.3-0.9 mm interventional wire to pass through. Optionally, the positioning wire is configured to locate the coronary sinus. A vascular delivery system (30) then tracts over the positioning wire to the intended treatment location.

In other embodiments, the vascular anchor (12) is made of a plastically deformable material such as stainless steel, cobalt chromium, or similar materials. In some embodiments, the vascular anchor (12) is made from a polymeric material such as PET or other plastically deformable material. In some embodiments, the deformable anchor (12) includes a collapsed deployment configuration and an expanded implanting configuration. In the collapsed deployment configuration, the anchor (12) is collapsed around a deflated endovascular balloon, as shown in FIG. 3B. The endovascular balloon may be made of any of the materials known to one with ordinary skill in the current state of the art, including PET or Mylar, polyurethane, or similar materials. In some embodiments (not shown in FIG. 3B), the endovascular balloon is defined by an annular inflation space with an internal diameter that is configured to allow passage of an interventional wire. In some embodiments, the vascular anchor (12) is collapsed on the annular balloon and held in a collapsed state by an outer sheath (32′). The delivery system (30′) including the outer sheath (32′), the annular balloon (36′) and a balloon catheter (34′), the vascular anchor (12), and the positioning wire is delivered into the coronary sinus. (Similar to descried above, the positioning wire is used to locate the coronary sinus.) Once the vascular anchor is implanted, the outer sheath (32′) is removed or partially retracted, exposing the vascular anchor (12), along with the annular balloon (36′). The balloon is then inflated, causing the vascular anchor (12) to deform, expand, and engage the inner surface of the vasculature.

As shown in FIG. 4, according to some embodiments, the vascular anchor (12) has a generally tubular profile with a hollow surface structure, which allows an easy radial expansion of the vascular anchor (12) during the deployment. The vascular anchor (12) includes a distal end (42), a proximal end (44), and a longitudinal lumen (46), which does not impede blood flow.

According to some embodiments, the overall length of the vascular anchor (12) in its deployed profile is 15-25 mm According to some embodiments, the general size of the vascular anchor (12) is 12-18 mm in diameter. According to some embodiments, the vascular anchor (12) has a generally tubular deployed profile. In another embodiment, the vascular anchor (12) has a conical or frustro-conical deployed configuration with its proximal end having a larger general diameter, and the distal end having a relatively smaller diameter. One skilled in the art should understand that the overall size of a vascular anchor (12) is designed for even distribution of the force to the surrounding tissue. As a result, it could vary based on an individual patient.

In another embodiment, the proximal end of the anchor is positioned approximately to the ostium (6) of the coronary sinus. In another embodiment, the proximal end of the vascular anchor (12) is positioned significantly into the coronary sinus. In a particular embodiment, the vascular anchor (12) is advanced until it resides along the posterior aspect of the left heart approximately 20-80 mm into the coronary sinus, for example the proximal end of the anchor is positioned 15 mm from the ostium and inside the coronary sinus. A clinician should determine the optimum implantation location based on each patient's symptom and anatomy. Thus, what has been disclosed here is merely an example, and should not be viewed as limiting.

In some embodiments of the present teachings, the vascular anchor (12) has a hollow surface structure along its tubular surface. As illustrated, each hollowed surface structure is separated by struts with wavy or zigzag patterns. A wavy and zigzag pattern allows the tubular body of the anchor to expand radially. According to some embodiments, the vascular anchor (12) with a hollow surface structure is fabricated by laser-cutting or acid-etching a pattern into a preformed tube, then shape-setting the anchor to the intended deployed configuration. In such embodiments, the vascular anchor (12) with a hollow surface structure is formed by slotting a hollow tube, for example, with a machining laser, a water drill, or other methods, and expanding the slotted hollow tube to form an open structure. Alternatively, a vascular anchor (12) with a hollow surface structure can be formed with a woven, knitted, or braided tubular metallic fabrics made out of metallic strands. The term “strand” used herein can be wires, cords, fibers, yarns, filaments, cables, threads, or the like, and these terms may be used interchangeably.

A vascular anchor (12) of the present teachings is configured to engage the surrounding tissues when the vascular anchor (12) is deployed. According to one embodiment, once exiting from the delivery sheath, the vascular anchor (12) expands radially so that it secures itself to the surrounding tissues. In some embodiments, the vascular anchor (12) is made of a thermal shape memory material so that once exposed inside the blood stream, the vascular anchor (12) expands radially by itself. In another embodiment, the vascular anchor (12) is expanded by a vascular balloon.

Once deployed inside a vasculature, the vascular anchor (12) expands and secures itself at a location inside the coronary sinus without migrating along the length of the vein. In some embodiments, the vascular anchor (12) secures to the surrounding tissues through interference between its tubular surface and the vasculature. In another embodiment, the vascular anchor (12) has tissue engagement features such as barbs, hooks. In some embodiments, the vascular anchor (12) is designed to locally expand the coronary sinus significantly. In some embodiments, the vascular anchor (12) when deployed causes the internal diameter of the vascular anchor (12) to increase by 50%. Due to the anatomical structure of the heart, it is sometimes preferred that the tissue engagement feature is oriented facing inward of the heart.

In some embodiments, the vascular anchor (12) is configured to engage the internal diameter of the coronary sinus near the coronary sinus ostium (6). In some embodiments, the vascular anchor (12) is configured to engage the Eustachian valve near the coronary sinus ostium (6). In some embodiments, the vascular anchor (12) completely punctures the ridge of tissue separating the coronary sinus ostium (6) from the right atrium. This ridge of tissue may be referred to as a Eustachian valve. In patients with significant tricuspid regurgitation, this tissue ridge is often enlarged or at least more pronounced by the dilation of the surrounding heart chambers. In yet other embodiments, the vascular anchor (12) is implanted into one of the venous branches extending from the coronary sinus.

As shown in FIG. 5, the deployed vascular anchor (12) is attached with a flexible tensioning member (18). The distal end of the flexible tensioning member (18) joins the vascular anchor (12). The proximal end of the flexible tensioning member (18) extends proximally and eventually exits to the outside of the body and remains in the control of a clinician. As shown in FIG. 5, before the treatment is completed, a portion of the flexible tensioning member (18) is exposed inside the right atrium. According to some embodiments, the flexible tensioning member (18) attaches to a proximal end of the vascular anchor (12). According to an alternative embodiment, the flexible tensioning member (18) attaches to any portion of the anchor. One skilled in the art should understand that the connection between the flexible tensioning member (18) and the vascular anchor (12) should be strong enough to withstand a certain amount of strain applied later. In some embodiments the vascular anchor (12) is made from a laser cut Nitinol tube which has been formed into a stent-like structure. An eyelet may be cut into the stent like structure, allowing the attachment of a suture material to the eyelet by a simple knot. In other embodiments, a suture is attached to the vascular anchor (12) by swaging, crimping, gluing, pinching, or clamping the suture to a feature of the vascular anchor (12).

FIG. 5 illustrates an embodiment of the present teachings, where the flexible tensioning member (18) attachment point is at or approximately to the ostium (6) of the coronary sinus. As shown, the flexible tensioning member (18) attaches to the vascular anchor (12) on its outside edge of the curve, further away from the tricuspid valve.

Now referring to FIGS. 6-8, where an annulus anchor (14) is deployed at a location along the tricuspid annulus (8). As shown in FIG. 8, the annulus anchor (14) has a distal portion (52), which is deployed against the right ventricle side of the annulus, a center portion (54), which is positioned through the annulus (8), and a proximal portion (56), which is positioned inside the right atrium. A second flexible tensioning member (16) attaches to the proximal portion of the annulus anchor (14). The flexible tensioning member (16) also has a distal end attaching to the annulus anchor (14), and a proximal end that extends proximally through the annulus anchor delivery system (60) and eventually outside of the body, and remains in control by a clinician. As shown in FIG. 8, once the annulus anchor (14) is deployed at a treatment location, a portion of the second flexible tensioning member (16) is exposed inside the right atrium.

Deployment of the annulus anchor (14) starts with an annulus anchor delivery system (60) gaining access to the right atrium. FIG. 6 illustrates an embodiment where the access to the right atrium is gained by the annulus anchor delivery system (60) entering the jugular vein, and extending through the right brachiocephalic vein and the superior vena cava, and reaching the right atrium. Alternative accesses to the right atrium could be gained through a standard right heart catheterization procedure, similar to those described above, for example, with the delivery system entering through the femoral vein, the inferior vena cava, and the right atrium.

Once inside the right atrium, the annulus anchor delivery system (60) further extends downwardly towards the tricuspid annulus (8). After an implant location is identified, a locating wire (68) punctures the annulus (8) to create an aperture. A tissue anchor is deployed at the location. Methods of locating and placing a locating wire (68) at a selected implant location is disclosed in U.S. patent application Ser. No. 14/190,732 (now U.S. Pat. No. 9,724,084), filed on Feb. 26, 2014, entitled “Devices and Methods for Percutaneous Tricuspid Valve Repair,” the content of which is incorporated by reference herein in its entirety.

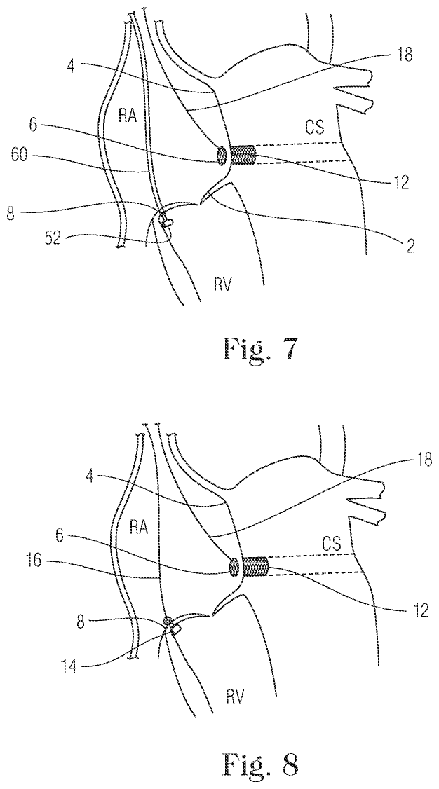

Once an implant location is identified and marked with a locating wire (68), as shown in FIG. 6, a locating wire (68) extends from the right atrium, across the tricuspid annulus (8), and into the right ventricle. An annulus anchor (14) is then deployed across the aperture created by the locating wire (68). Unlike the deployment of the vascular anchor (12), the annulus anchor (14) is deployed in a step-wise process. As the distal end of the annulus anchor delivery system (60) extends through the aperture into the right ventricle, the distal end of the annulus anchor (14) is pushed outside of the distal end of the annulus anchor delivery system (60). The distal portion (52) of the annulus anchor (14) assumes its radially expanded profile, as shown in FIG. 7. The delivery system holds the rest of the annulus anchor (14), then is pulled proximally so that the distal portion (52) of the annulus anchor (14) is positioned against the annulus tissue. The delivery system (60) is then pulled further proximally to expose the center portion (54) of the annulus anchor (14), and the proximal portion (56) of the annulus anchor (14).

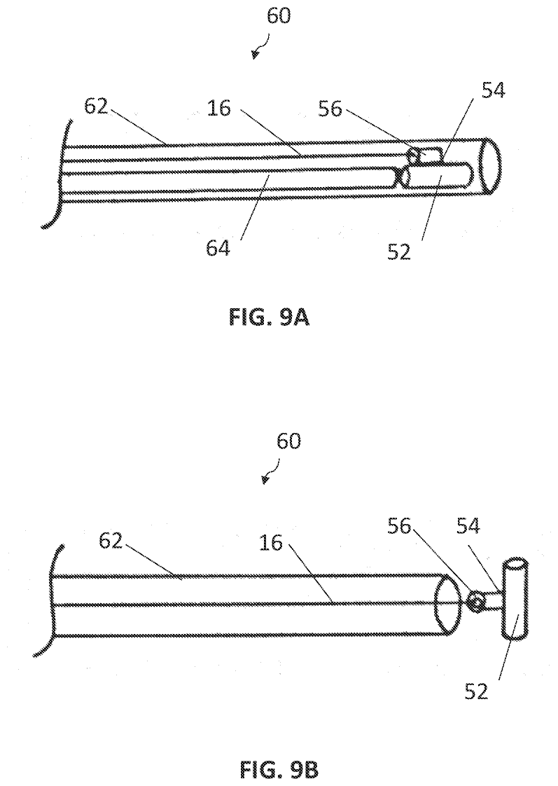

FIGS. 9A-9B illustrate an embodiment of an annulus anchor (14) of the present teachings. As shown in FIG. 9B, the annulus anchor (14) has a large elongated distal portion (52), which is designed to be pivotable at the location where it joins the center portion (54) of the annulus anchor (14). During delivery (as shown in FIG. 9A), the distal portion (52) of the anchor (14) pivots radially so that it aligns with the center portion (54) of the anchor (14). The entire annulus anchor (14) is configured to be slidably disposed inside the delivery sheath (62). FIG. 9A further illustrates that the annulus anchor delivery system (60) also includes an outer sheath (62) and an inner catheter (64) which resides inside the lumen of the outer sheath (62). The distal end of the inner catheter (64) contacts the annulus anchor (14) and is configured to push the annulus anchor (14) distally, or prevent annulus anchor (14) from sliding proximally inside the delivery sheath (62). According to some embodiments, simply unsheathing the delivery sheath (62), or pushing the delivery catheter (64) distally while holding the delivery sheath (62) steady allows the annulus anchor (14) to be exposed and expand.

According to some embodiments, upon deployment, the distal portion (52) of the annulus anchor (14) pivots automatically and assumes its profile perpendicular to the center portion (54) of the anchor (14), as shown in FIG. 9B. Upon deployment, the distal portion (52) of the annulus anchor (14) is positioned against the ventricular side of the annulus. According to some embodiments of the present teachings, the deployed annulus anchor (14) is deployed at the anterior-posterior commis sure or 3 mm in each direction of this commissure.

The center portion (54) of the annulus anchor (14) also has an elongated profile. The center portion (54) of the annulus anchor (14) pivotably joins the distal portion (52) of the anchor (14), and, in some embodiments, pivotably attaches to the proximal portion (56) of the anchor (14). As shown in FIG. 9A, during delivery, the center portion (54) of the anchor (14) aligns with the distal portion (52) of the anchor (14), and is slidably disposed inside the delivery sheath (62). During deployment, at least a portion of, sometimes the entire, center portion (54) of the annulus anchor (14) is also deployed along with the distal portion (52) of the anchor. Specifically, as the delivery system (60) is pulled back proximally, and the distal portion (52) of the anchor (14) is positioned against the annulus, the center portion (54) of the anchor (14) is positioned through the aperture across the annulus (8).

According to some embodiments, the proximal portion (56) of the anchor (14) is an extension of the center portion (54) of the anchor (14). Thus, the connection between the center portion (54) and the proximal portion (56) is rigid. One skilled in the art should understand, for example, that the proximal portion (56) of the anchor (14) can adopt other profiles, for example, those similar to the profiles of the distal portion (52). Once released from the delivery system (60), the proximal portion (56) of the annulus anchor (14) can also pivot and assume its expanded profile, sometimes similar to the distal portion (52) of the annulus anchor (14).

Many other shapes and profiles could be adopted for the purpose of this application, including, for example, the annulus anchors (14) disclosed in U.S. patent application Ser. No. 12/273,670 (now U.S. Pat. No. 8,951,286), filed on Nov. 19, 2008, entitled “Tissue Anchor and Anchoring System,” U.S. patent application Ser. No. 11/174,951 (now U.S. Pat. No. 8,951,285), filed on Jul. 5, 2005, entitled “Tissue Anchor, Anchoring System and Methods of Using the Same,” U.S. patent application Ser. No. 13/777,042 (now U.S. Pat. No. 9,259,218), filed on Feb. 26, 2013, entitled “Tissue Anchor and Anchoring System,” each of which is incorporated by reference herein in its entirety. One skilled in the art should also understand that examples of suitable tissue anchors include, but not be limited to, tissue fasteners, tissue pledgets, or tissue staples etc.

As shown in FIG. 8, the proximal portion (56) of an annulus anchor (14) joins a second flexible tensioning member (16). During delivery, the second flexible tensioning member (16) is slidably disposed within the annulus anchor delivery system (60). As shown in FIG. 8, the distal end of the flexible tensioning member (16) joins the proximal portion (56) of the annulus anchor (14). The proximal end of the flexible tensioning member (16) extends proximally through the delivery system (60) and eventually to the outside of the body, and remains in the control of a clinician. As shown in FIG. 8, before the treatment is completed, a portion of the second flexible tensioning member (16) is located inside the right atrium.

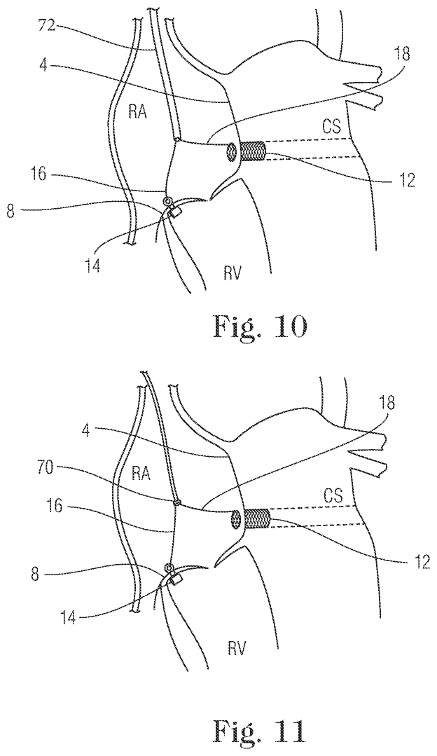

FIGS. 10-11 illustrate an embodiment of a plication procedure of the present teachings, for example, for reducing the circumference of a tricuspid valve annulus (8). Upon deployment of a vascular anchor (12) and an annulus anchor (14) at their intended locations according to the present teachings, the free ends of the tensioning members (16,18) from each anchor (12, 14) are pulled so that the anchors will be drawn towards each other to plicate the tricuspid annulus (8), as shown in FIG. 10. According to some embodiments, once both the vascular and annulus anchors (12, 14) are deployed at their respective locations, there is a first natural distance between the anchors (12, 14). Upon applying tension to both the tensioning members (16, 18) extending from the anchors (12, 14), the distance between the two anchors (12, 14) reduces to a second distance. Doing so, the natural circumference of the tricuspid annulus (8) is reduced. A crimp or other lock member (70) may then be delivered through a lock member delivery system (72) and be used to lock in the desired circumference reduction by crimping onto the tensioning members (16, 18). Upon deploying a lock member (70) in place, the excess tensioning member proximal to the lock member (70) may then be cut and removed from the body. In some embodiments, the reduction from the first natural distance to the second reduced distance is around 25-35 mm. For example, before tension is applied, the distance between both the anchors can be 25-35 mm Upon tensioning, the distance between the two anchors is between 0-10 mm.

According to some embodiments, as shown in FIG. 11, the lock member (70) and both the tensioning members (16, 18) are located inside the right atrium after the procedure. Since the coronary sinus drains into the right atrium, and its opening locates between the inferior vena cava and the tricuspid valve on the inferior aspect of the atrial septum (4), by pulling the anterior-posterior commissure toward the septum, in some embodiments, the tricuspid annulus (8) changes it natural shape, resulting in an improved cooptation between the anterior and posterior leaflets with the septal leaflet. In some instances, the plication achieved by the two anchors may bicuspidize the valve, creating two functioning leaflets. In some instances, the plication completely eliminates the posterior leaflet. In some instance, the plication eliminates the posterior leaflet and shortens the effective length of the septal leaflet, for example, the plication may completely obviate the posterior leaflet and may reduce the functional length of the septal leaflet by 10-15 mm.

Suitable lock members include those known in the art and those described in U.S. application Ser. No. 11/753,921 (which published as US 2007/0276437), filed on May 25, 2007, entitled “Lockers for Surgical Tensile Members and Methods of Using the Same to Secure Surgical Tensile Members,” the entire disclosure of which is incorporated herein by reference. With the tensioning members secured by a lock member (not shown), the excess tensioning member(s) proximal to the lock member can be removed by a cutter, including, for example, a cutter disclosed in U.S. patent application Ser. No. 11/935,054 (now U.S. Pat. No. 8,911,461), filed on Nov. 5, 2007, entitled “Suture Cutter and Method of Cutting Suture,” the entire disclosure of which is incorporated herein by reference.

Upon the deployment of the lock member (70) to lock the tensioning members in place, the circumference of the tricuspid annulus (8) is then reduced by some first reduction amount. The reduced tricuspid valve annulus therefore has lower regurgitation and allows the body to remodel.

According to some embodiments, the tensioning member (18) joins the distal end (42) of the vascular anchor (12), such as shown and described herein. One skilled in the art should understand that the tensioning member (18) could also join the proximal end (44) of the vascular anchor (12). In some embodiments, where a vascular anchor (12) joins the tensioning member (18) at its distal end (42), the reduced tricuspid annulus (8) valve reduces and/or eliminates valve regurgitation. As the body remodels, the right atrial pressure and coronary sinus venous pressure should decrease with the decreased tricuspid valve regurgitation. This positive remodeling allows the previously dilated coronary sinus to shrink in diameter. According to some embodiments of the present teachings, the reduction in diameter of the coronary sinus causes the vascular anchor (12) to elongate, thereby reducing its diameter. As the vascular anchor (12) elongates, its distal end (42) extends further distally to the inside of the coronary sinus, applying additional tension to the tensioning member (18) connected at its distal end. This could supply additional distance reduction between the vascular anchor (12) and annulus anchor (14), thereby plicating the annulus (8) further. This effect further reduces the circumference of the tricuspid annulus (8), and further accelerates the reduction of the tricuspid valve regurgitation. According to some embodiment of the present teaching, the vascular anchor is designed with a pre-set tension limiting mechanism. Once the pre-set tension limit is reached, for example during implantation or sometimes triggered by post procedure coronary sinus remodeling, the vascular anchor will change its shape/form in part or in whole to prevent further tension increase. For example, the proximal portion of the vascular could elongate under excess tension and thereby extend proximally.

FIGS. 12-14 illustrate another embodiment of the present teachings, where both vascular anchor (12) and annulus anchor (14) are joined with one tensioning member (18). In this embodiment, the implantation of the vascular anchor (12) is the same as described before, for example, in relation to FIGS. 2-5. Similar to what has been described, for example, in relation to FIG. 6, a position wire (68) is then placed at a location across the tricuspid annulus (8). With the proximal end of the tensioning member (18) extending from the vascular anchor (12) to the outside of the body. A clinician then slides an annulus anchor (14) over the proximal end of the tensioning member (18), transforms the annulus anchor (14) into its collapsed delivery profile, and assembles it to the distal end portion of the annulus anchor delivery system (60). The annulus anchor delivery system assembly is similar to what has been described above for example in relation to what has been described in FIGS. 9A-9B. This assembly then tracks over the positioning wire (68) and advances distally toward the tricuspid annulus (8) implantation location as shown in FIG. 12.

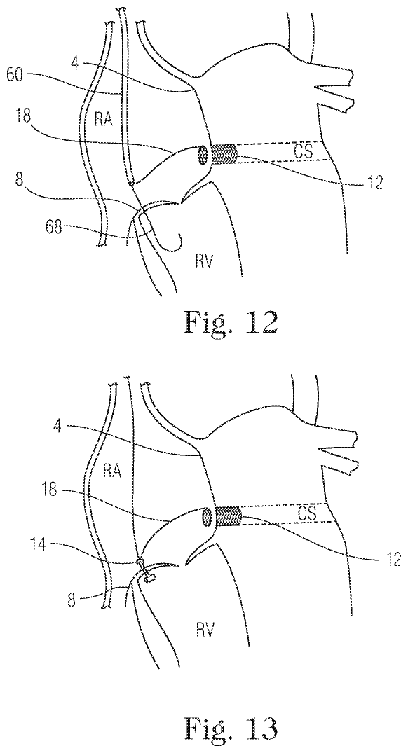

Continuing referring to FIG. 13, an annulus anchor (14) is then deployed across the tricuspid annulus (8) with its distal portion (52) positioned against the annulus (8) inside the right ventricle, its center portion (54) positioned across the annulus (8), and its proximal portion (56) inside the right atrium. As shown in FIG. 13, the tensioning member (18) has its distal ends fixedly attached to the vascular anchor (12). The annulus anchor (14) has its proximal end slide over the tensioning member (18). By applying tension to the tensioning member (18), the natural distance between the annulus anchor (14) and the vascular anchor (12) is reduced, causing the annulus to plicate and/or bicuspidize. The circumference of the tricuspid annulus (8) is thereby reduced, and tricuspid regurgitation is also reduced.

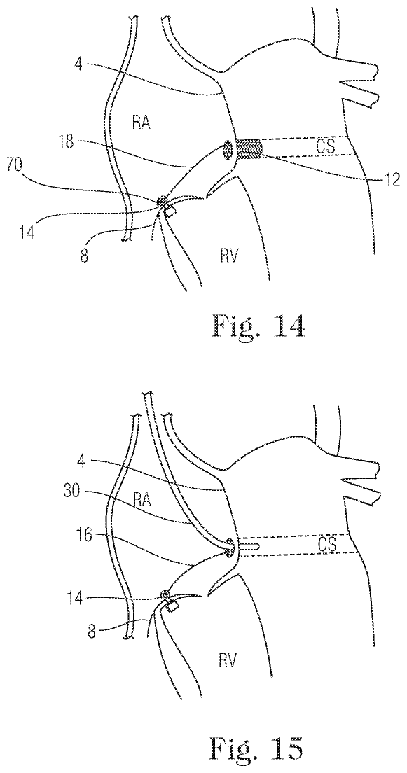

Upon removing both the position wire (68) and annulus anchor delivery system (60), a tension can be applied by a clinician to the tensioning member (18), thereby pulling two anchors (12, 14) closer to each other, and effectively reducing the circumference of the tricuspid annulus (8). Then, a clinician slides a lock member (70) over the tensioning member's proximal end. Using a lock delivery system similar to what has been described above, a lock member (70) is then deployed against the annulus anchor (14), holding the pre-loaded tension on the tensioning member, maintaining the desired circumference reduction on the tricuspid annulus (8). Upon completion of the procedure, the excess tensioning member is then cut and removed from the body. FIG. 14 illustrates a vascular anchor (12) deployed inside the coronary sinus, an annulus anchor (14) deployed across the tricuspid annulus (8), and one tensioning member connecting both anchors with a lock member (70) maintaining the shortened distance between the two anchors (12, 14).

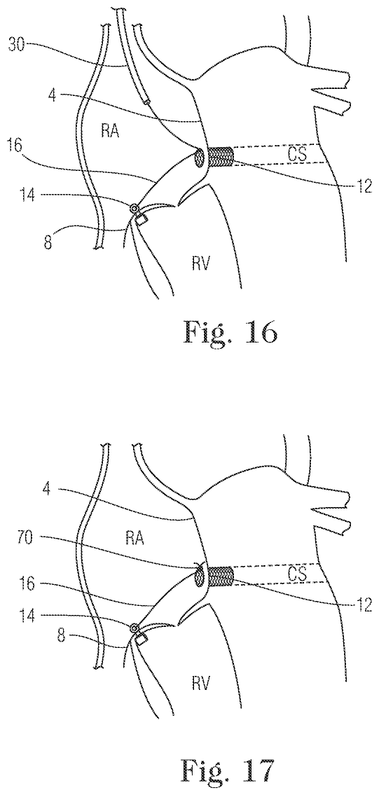

FIGS. 15-16 further illustrate another embodiment of the present teachings. Similar to the embodiments described in accordance with FIGS. 12-14, the present embodiment also involves a vascular anchor (12) and an annulus anchor (14) joined by one tensioning member (16). In this embodiment, the annulus anchor (14) is deployed first at an identified treatment location. The procedure and methods of identifying an implantation location and deploying an annulus anchor (14) are similar to what has already been described, for example, in accordance with FIGS. 6-8.

With an annulus anchor (14) deployed across tricuspid annulus (8), a tensioning member (16) fixedly joins to the annulus anchor (14) at its proximal end, and extends proximally outside of the body. With the proximal end of the tensioning member (16) extending to the outside of the body, a clinician then slides a vascular anchor (12) over the proximal end of the tensioning member (16), then transforms the vascular anchor (12) into its collapsed delivery profile and assembles it to the distal end portion of the vascular anchor delivery system (30). The vascular anchor delivery system assembly is similar to what has been described above, for example in relation to what has been described in FIGS. 3A-3B. Optionally, this assembly then tracks over the positioning wire that is put in place inside the coronary sinus. FIG. 15 illustrates that a vascular anchor delivery system assembly carrying the vascular anchor (12) advances distally, and is placed inside the coronary sinus implantation location.

The vascular anchor (12) is then deployed in accordance with what has been disclosed above. Upon deployment, as shown in FIG. 16, an annulus anchor (14) is positioned across the annulus, a vascular anchor (12) is positioned inside the coronary sinus, and a tensioning member (16) fixedly joins the annulus anchor (14) at its distal end and slides through at least a portion of the vascular anchor (12). The proximal end of the tensioning member (16) extends through at least a portion of the vascular anchor (12), and extends proximally to the outside of the body.

At this point, a tension is applied by a clinician to the tensioning member (16), thereby pulling two anchors (12, 14) closer to each other and effectively reducing the circumference of the tricuspid annulus (8). A clinician then slides a lock member (70) over the proximal end of the tensioning member (16), and uses a lock delivery system similar to what has been described above to deploy a lock member (70) against the vascular anchor (12), holding the pre-loaded tension on the tensioning member (16), maintaining the desired circumference reduction on the tricuspid annulus (8). Upon completion of the procedure, the excess tensioning member (16) is then cut and removed from the body. FIG. 17 illustrates a vascular anchor (12) deployed inside the coronary sinus, an annulus anchor (14) deployed across the tricuspid annulus (8), and one tensioning member connecting both anchors with a lock member (70) maintaining the shortened distance between the two anchors (12, 14).

Although FIGS. 1-17 illustrate embodiments of the tricuspid annulus plication system with one vascular anchor and one annulus anchor, one skilled in the art should understand that a system with one vascular anchor and more than one annulus anchor can also be applied in the treatment, and therefore such systems and uses thereof are within the scope of present teachings.

According to some embodiments, the vascular anchor has a general tubular profile, as illustrated in the figures. The vascular anchor can also have a general conical profile as disclosed above with its proximal end having a larger profile than its distal end. One skilled in the arts should understand that the vascular anchor could take on other profile that is suitable for deploying inside the coronary sinus.

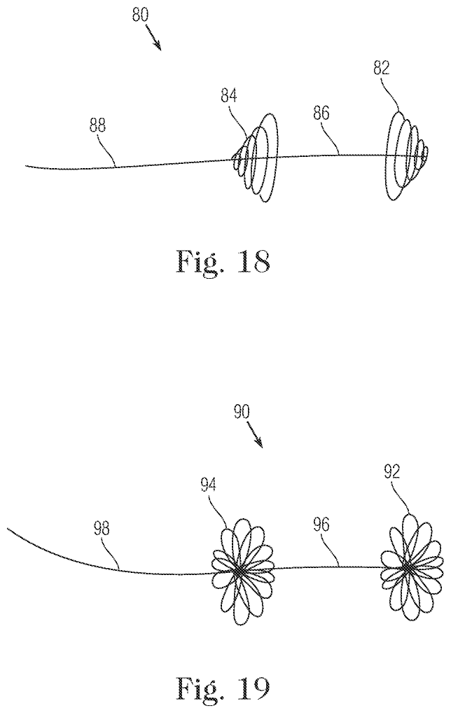

FIGS. 18-19 illustrate other embodiments of a vascular anchor (80, 90) of the present teachings. As illustrated, the vascular anchor (80, 90) has a radially enlarged distal portion (82, 92), a radially enlarged proximal portion (84, 94), and an elongated member (86, 96) connecting both the distal and proximal portions (82, 84, 92, 94). The enlarged distal portion (82, 92) could be in a shape of a cone, a disc, or other suitable profile. Similarly, the enlarged proximal portion (82, 92) could be in a shape of a cone, a disc, or other suitable profile. The elongated member (86, 96) has a smaller radial profile comparing to the distal and proximal portions (82, 84, 92, 94) of the vascular anchor (12). The elongated member (86, 96) connects the distal portion (82, 92) at its distal end, the proximal portion (84, 94) of the vascular anchor (80, 90) at its proximal end.

According to some embodiments, the vascular anchor (80, 90) as shown in FIGS. 18-19 are made of one continuous wire. FIG. 18 illustrates an embodiment of the present teachings where the distal portion (82) of the wire forms a roughly spiral shape. The wire continues from the distal portion (82) of the vascular anchor (80), forms the elongated member (86), and continues toward the proximal portion (84). The proximal portion (84) of the wire also forms a roughly spiral shape. FIG. 19 illustrates another embodiment of the present teachings where, the distal and proximal portions (92, 94) of the wire forms a series of semi-circular turns which are configured to expand the inner diameter of the coronary sinus, the middle portion of the wire forms the elongated member (96) connecting the distal (92) and proximal portions (94) of the vascular anchor (90). One skilled in the art should understand FIGS. 18-19 are merely illustration of two embodiments, the vascular anchor formed of a wire can adopt other shapes and profiles for its distal and proximal portion. And the distal and proximal portions of such vascular anchor can have the same or different profiles.

In some embodiments, the wire used to form such vascular anchor is a super-elastic shape-memory wire. The shape memory wire may be pre-set into a series of large loops. The vascular anchor is delivered in a collapsed profile and once exposed inside the coronary sinus, it resumes it pre-set profile. In some embodiments, the shape memory wire has a diameter of 0.3-0.6 mm.

In one embodiment, a tensioning member (88, 98) joins the distal portion (82, 92) of the vascular anchor (80, 90). In another embodiment, a tensioning member (88, 98) joins the proximal portion of the vascular anchor (80, 90). Yet in another embodiment, the wire that forms the vascular anchor (80, 90) becomes a tensioning member (88, 98) that joins the vascular anchor and is used for tensioning the device.

According to various embodiments of the present teachings, a radiopaque marker or textured surface is used to make the device visible by using a radiographic imaging equipment such as an X-ray, magnetic resonance, ultrasound or other imaging technique. A marker disclosed herein may be applied to any part of the guide, catheter, or devices disclosed in present teachings. A radiopaque marker can be sewed, adhered, swaged riveted, or otherwise placed and secured on the guide, catheter, and/or devices. The radiopaque marker may be made from a material selected from tantalum, tungsten, platinum, irridium, gold, an alloy thereof, or another material known to those with ordinary skill in the art. The radiopaque marker can also be made from cobalt, fluorione, or another paramagnetic material, or another MR visible material known to those with ordinary skill in the arts. Additionally, a contrast media injected into the atrium, ventricle, or artery may also be used to confirm the positioning under a fluoroscope.

Exemplary methods for treating tricuspid valve regurgitation described herein comprises a number of other steps. One skilled in the art should understand that the sequence of the steps can be changed, or each of steps can be omitted or modified according to each patient's needs. And those modifications should also be considered as within the scope of the present teachings. For example, access to the right atrium is gained by entering the jugular vein according to some embodiments described herein, but one skilled in the art should understand that access to the right atrium can also be achieved by entering the femoral vein and through the inferior vena cava (IVC). In addition, although the tensioning member and tissue anchor, as well as the tensioning member and vascular anchor are described as separate components according to some embodiments, one skilled in the art should understand that the tensioning member and each of the anchor can be part of an integral part. In another example, although the lock member described or incorporated above is a component separate from the tensioning member, one skilled in the art should understand other types of locking mechanisms can also be incorporated, including, for example, a knot that is part of the tensioning member and self-tightens as the tensioning member is pulled by a clinician. The present teachings also disclose certain exemplary delivery catheters/sheathes for delivering a tissue anchor, a vascular anchor, or/and a lock, and for removing a part of a tensioning member. A person skilled in the art should understand that some or all of the delivery catheters/sheathes can be combined, all of which are within the scope of this disclosure. Thus, any of the embodiments described herein should not be used to limit the scope of the invention.

Unless otherwise defined, all technical and scientific terms used herein have the same meaning as commonly understood by one of ordinary skill in the art to which this present teachings belong. Methods and materials similar or equivalent to those described herein can be used in the practice of the present teachings. In case of conflict, the specification, including definitions, controls. In addition, the materials, methods, and examples are illustrative only and not intended to be limiting.

1. A method of treatment at a tricuspid valve of a heart of a subject, the tricuspid valve having an annulus and being disposed between a right atrium and a right ventricle of the heart, the heart having a coronary sinus, and the method comprising:
transluminally advancing a first anchor to the coronary sinus;
anchoring the first anchor inside the coronary sinus;
transluminally advancing a second anchor to the right atrium;
anchoring the second anchor at a site that is across the annulus from the coronary sinus by driving a distal portion of the second anchor from the right atrium into the annulus at the site;
subsequently to the step of anchoring the first anchor and the step of anchoring the second anchor:
reducing a distance between the first and second anchors by applying tension to a tensioning member coupled to the first anchor and the second anchor; and
maintaining the tension by locking a lock member to the tensioning member.

2. The method according to claim 1, further comprising, prior to anchoring the second anchor, forming an aperture at the site by puncturing the annulus with a wire, and wherein driving the distal portion into the annulus at the site comprises driving the distal portion through the aperture.

3. The method according to claim 1, wherein the tricuspid valve has an anterior leaflet, a septal leaflet, and a posterior leaflet, and wherein reducing the distance between the first and second anchors comprises reducing the distance between the first and second anchors such that the posterior leaflet becomes in effect eliminated.

4. The method according to claim 1, wherein:
advancing the first anchor comprises advancing the first anchor while the first anchor is collapsed around a deflated balloon of a balloon catheter, and
anchoring the first anchor comprises plastically expanding the first anchor within the coronary sinus by inflating the balloon.

5. The method according to claim 1, wherein advancing the second anchor to the right atrium comprises transluminally advancing the second anchor to the right atrium while the second anchor is disposed within a delivery device.

6. The method according to claim 1, wherein:
the tensioning member is a single tensioning member;
reducing the distance comprises reducing the distance by applying tension to the single tensioning member; and
maintaining the tension comprises maintaining the tension by locking the lock member to the single tensioning member.

7. The method according to claim 1, wherein anchoring the first anchor comprises anchoring the first anchor inside the coronary sinus while the tensioning member is coupled to the first anchor.

8. The method according to claim 1, wherein locking the lock member to the tensioning member comprises locking the lock member to the tensioning member subsequently to reducing the distance between the first and second anchors.

9. The method according to claim 1, wherein anchoring the second anchor at the site comprises anchoring a single second anchor to the site.

10. The method according to claim 1, further comprising sliding the lock member along the tensioning member to the right atrium subsequently to anchoring the second anchor at the site.

11. The method according to claim 10, wherein applying tension to the tensioning member comprises applying tension to the tensioning member subsequently to sliding the lock member along the tensioning member to the right atrium.

12. The method according to claim 1, wherein anchoring the first anchor inside the coronary sinus comprises anchoring the first anchor inside the coronary sinus prior to anchoring the second anchor at the site.

13. The method according to claim 12, wherein anchoring the first anchor inside the coronary sinus comprises anchoring the first anchor inside the coronary sinus prior to transluminally advancing the second anchor to the right atrium.

14. The method according to claim 13, wherein anchoring the first anchor inside the coronary sinus comprises anchoring the first anchor inside the coronary sinus with the tensioning member coupled to the first anchor, and wherein transluminally advancing the second anchor to the right atrium comprises sliding the second anchor over the tensioning member toward the right atrium.

15. The method according to claim 14, wherein locking the lock member to the tensioning member comprises locking the lock member to the tensioning member such that the second anchor is disposed, on the tensioning member, between the lock member and the first anchor.

16. The method according to claim 1, wherein:
the tensioning member is a second tensioning member, and is coupled to the first anchor via a first tensioning member,
reducing the distance comprises reducing the distance by applying tension to the first and second tensioning members, and
maintaining the tension comprises maintaining the tension by locking the lock member to the first and second tensioning members.

17. The method according to claim 16, wherein locking the lock member to the first and second tensioning members comprises locking the lock member to the first and second tensioning members such that the lock becomes implanted in the right atrium.

18. The method according to claim 1, wherein:
transluminally advancing the first anchor to the coronary sinus comprises transluminally advancing the first anchor to the coronary sinus while the first anchor is maintained in a collapsed state within the first delivery device, and
anchoring the first anchor comprises radially expanding the first anchor within the coronary sinus.

19. The method according to claim 18, wherein anchoring the first anchor comprises ejecting the first anchor from the first delivery device within the coronary sinus such that the first anchor self-expands within the coronary sinus.
